(19) United States
(12) Patent Application Publication
(10) Pub. No.: US 2010/0104566 A1
(43) Pub. Date: Apr. 29, 2010

(54) ANTITUMOR COMBINATION COMPRISING A MORPHOLINYL ANTHRACYCLINE AND AN ANTIBODY

(75) Inventor: Maria Cristina Geroni, Milan (IT)

Correspondence Address: SCULLY SCOTT MURPHY & PRESSER, PC, 400 GARDEN CITY PLAZA, SUITE 300, GARDEN CITY, NY, 11530, US

(73) Assignee: NERVIANO MEDICAL SCIENCES S.R.L., Nerviano (MI) (IT)

(21) Appl. No.: 12/524,984

(22) PCT Filed: Jan. 24, 2008
(86) PCT No.: PCT/EP2008/050834

§ 371 Date: Jul. 29, 2009

(30) Foreign Application Priority Data
Jan. 29, 2007 (EP) 07101325.4

(51) Int. Cl. A61K 39/395; A61P 35/00

(52) U.S. Cl. 424/133.1; 424/158.1; 424/142.1; 424/141.1

(57) ABSTRACT

The present invention provides the combined use of a morpholinyl anthracycline derivative of formula (I) or a pharmaceutically acceptable salt thereof, such as nemorubicin hydrochloride, and an antibody inhibiting a growth factor or its receptor, in the treatment of tumors. Also provided is the use of the said combinations in the treatment or prevention of metastasis or in the treatment of tumors by inhibition of angiogenesis.

The present invention relates to the field of cancer treatment and provides an antitumor combination comprising a morpholinyl anthracycline derivative and an antibody inhibiting a growth factor or its receptor with antineoplastic effect.

Morpholinyl anthracyclines are known in the art as cytotoxic agents useful in antitumor therapy, see U.S. Pat. No. 4,672,057.

Cancers are a leading cause of death in humans; surgery, radiation and chemotherapy are the useful means to fight cancers. In particular, combined chemotherapy, designed to treat cancer by using more than one drug in combination or association, is a well-accepted modality of treatment of neoplastic diseases such as cancer. Several efforts have been and are still being undertaken in order to select antitumor combinations more and more active and safe to be administered to a patient suffering from a cancer. The increase of the antitumor efficacy of a known antitumor compound by administering the same in combination with one or more different antitumor drugs in order to reduce the toxic effects of the individual agents when used alone, and in some instances because the combination has greater efficacy than when either agent is used alone, is a strongly felt need in the field of anticancer therapy.

For example, WO 04/082579 and WO 00/066093 (Nerviano Medical Sciences Srl) are relating to combined use of morpholinyl anthracycline derivatives with radiotherapy or another anticancer drug such as an alkylating agent, an antimetabolite, a topoisomerase I or topoisomerase II inhibitor or a Pt derivative.

The present invention fulfils the need of improved cancer treatment by providing a combination or a combined administration of a morpholinyl anthracycline derivative or a pharmaceutically acceptable salt, with an antibody inhibiting a growth factor or its receptor having antineoplastic effect.

The present invention provides new combinations of a morpholinyl anthracycline derivative with known pharmaceutical agents that are particularly suitable for the treatment of proliferative disorders, especially cancer. More specifically, the combinations of the present invention are very useful in therapy as antitumor agents and lack, in terms of both toxicity and side effects, the drawbacks associated with currently available antitumor drugs.

It is therefore a first object of the present invention a combination comprising a morpholinyl anthracycline derivative having formula (I)

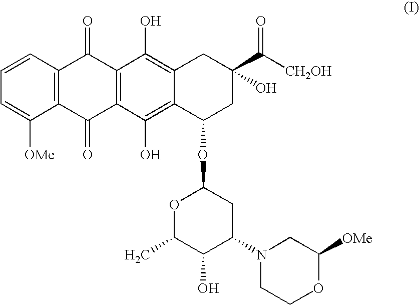

or a pharmaceutically acceptable salt thereof, and an antibody inhibiting a growth factor or its receptor.

Another aspect provides a pharmaceutical composition comprising a combination according the invention admixed with a pharmaceutically acceptable carrier, diluent or excipient.

A further aspect relates to a combination according the invention for treating a proliferative disorder. A still further aspect relates to a pharmaceutical product comprising a morpholinyl anthracycline as defined above and an antibody inhibiting a growth factor or its receptor, as a combined preparation for simultaneous, sequential or separate use in therapy. Another aspect relates to a method of treating a proliferative disorder, said method comprising simultaneously, sequentially or separately administering a morpholinyl anthracycline as defined above and an antibody inhibiting a growth factor or its receptor to a subject.

A still further aspect relates to the use of a morpholinyl anthracycline as defined above in the preparation of a medicament for the treatment of a proliferative disorder, wherein said treatment comprises simultaneously, sequentially or separately administering a morpholinyl anthracycline as defined above and an antibody inhibiting a growth factor or its receptor.

Another aspect relates to the use of a morpholinyl anthracycline as defined above and an antibody inhibiting a growth factor or its receptor in the preparation of a medicament for treating a proliferative disorder.

In the present description, unless otherwise specified, the morpholinyl anthracycline derivative having formula (I) is nemorubicin, chemical names (8S-cis, 2″S)-7,8,9,10-tetrahydro-6,8,11-trihydroxy-8-(hydroxyacetyl)-1-methoxy-10-{[2,3,6-trideoxy-3-(2-methoxy-4-morpholinyl)-α-L-lyxo-hexopyranosyl]oxy}-5,12-naphthacenedione and 3′desamino-3′[2(S)methoxy-4-morpholinyl]doxorubicin.

The term “pharmaceutically acceptable salt” refers to those salts retaining the biological effectiveness and properties of the parent compound. Such salts include acid addition salts obtained by reaction of the free base of the parent compound with inorganic acids such as hydrochloric, hydrobromic, nitric, phosphoric, sulfuric, and perchloric acid and the like; or with organic acids such as acetic, maleic, methanesulphonic, ethanesulfonic, tartaric, citric, succinic and the like.

Preferably, nemorubicin is in the form of its hydrochloride salt.

U.S. Pat. No. 4,672,057 discloses and claims nemorubicin, preparation process, pharmaceutical compositions and medical uses thereof.

In particular, nemorubicin represents a therapeutic option in the treatment of a liver cancer, and nemorubicin administration ways are described and claimed in WO 00/15203 and WO 04/75904.

Monoclonal antibodies (MoAbs) against growth factors or their receptors have been revealed to be effective therapeutic agents in antitumor therapy [see for a reference, Cancer Sci. 95: 621-25, (2004); Curr. Mol. Med 4: 539-47, (2004)].

Multiple mechanisms of monoclonal antibody action are being exploited for this purpose. Antibodies can sequester growth factors and prevent the activation of crucial growth factor receptors. A monoclonal antibody directed against the vascular endothelial growth factor (VEGF) has been shown to be a potent neo-vascularisation inhibitor (bevacizumab). An antibody against the extracellular domain of the epidermal growth factor (EGF) receptor prevents the binding of the ligand to the receptor and thereby its activation (cetuximab). EGFR activity, however, is absolutely required for the survival and proliferation of certain human tumour cells. An antibody which interferes with the dimerisation of the ErbB2 and the ErbB3 members of the EGF receptor family prevents the association of a most potent signaling module (pertuxumab). The signals emanating from this dimer determine many phenotypic properties of e.g. human breast cancer cells. A monoclonal antibody also directed against ErbB2 (an oncogene that encodes a receptor tyrosine kinase of the EGF-receptor family) has been most successful, clinically and commercially (trastuzumab). This antibody interferes with signals generated by the receptor and causes the arrest of the cell cycle in tumour cells. A selection of these agents is shown in Table 1.

TABLE 1
Antibodies inhibiting growth factors or
their receptors in clinical development

| Target | Name |
|---|---|
| VEGF | Bevacizumab |
| EGF-R | Cetuximab; Panitumumab; Matuzumab; Nimotuzumab |
| ErbB-2 | Trastuzumab; Pertuzumab |

It has now been surprisingly found that the antitumor effect of a morpholinyl anthracycline derivative of formula (I) as defined above is enhanced when it is administered in combination with an antibody inhibiting a growth factor or its receptor. In particular, the effect of the combined administration is additive or significantly increased (synergic effect) with respect to the effect obtained administering each drug as single agent.

According to a preferred embodiment of the invention, the antibody inhibiting growth factor or its receptor is Bevacizumab (antibody to vascular endothelial growth factor), Cetuximab, Panitumumab, Matuzumab, Nimotuzumab (antibodies to epidermal growth factor receptor), Trastuzumab or Pertuzumab (antibodies to ErbB2).

According to a more preferred embodiment of the invention, the antibody inhibiting growth factor or its receptor is Bevacizumab.

The term “pharmaceutically effective amount” shall mean that amount of a drug or pharmaceutical agent that will elicit the biological or medical response of a tissue, system, animal or human that is being sought by a researcher or clinician. This amount can be a therapeutically effective amount.

The term “therapeutically-effective” is intended to qualify the amount of each agent for use in the combination therapy, which will achieve the goal of improvement in disease severity and the frequency of incidence over treatment of each agent by itself, and/or of amelioration of adverse side effects typically associated with alternative therapies.

The combinations or combined preparations according to the present invention would be useful for the treatment of cancer. Preferably, the subject methods and compositions of the present invention may be used for the treatment of neoplasia disorders including benign, metastatic and malignant neoplasias, and also including acral lentiginous melanoma, actinic keratoses, adenocarcinoma, adenoid cystic carcinoma, adenomas, adenosarcoma, adenosquamous carcinoma, astrocytic tumors, bartholin gland carcinoma, basal cell carcinoma, bronchial gland carcinomas, capillary, carcinoids, carcinoma, carcinosarcoma, cavernous, cholangiocarcinoma, chondosarcoma, choriod plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic polypeptide, papillary serous adenocarcinoma, pineal cell, pituitary tumors, plasmacytoma, pseudosarcoma, pulmonary blastoma, renal cell carcinoma, retinoblastoma, rhabdomyosarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiated carcinoma, uveal melanoma, verrucous carcinoma, vipoma, well differentiated carcinoma, and Wilm's tumor.

The terms “treating” or “to treat” mean to alleviate symptoms, eliminate the causation either on a temporary or permanent basis, or to prevent or slow the appearance of symptoms. The term “treatment” includes alleviation, elimination of causation of or prevention of cancer. Besides being useful for human treatment, these combinations are also useful for treatment of mammals, including horses, dogs, cats, rats, mice, sheep, pigs, etc.

The term “subject” for purposes of treatment includes any human or animal subject who is in need of the prevention of, or who has cancer, cardiovascular disease, or pain, inflammation and/or any one of the known inflammation-associated disorders. The subject is typically a mammal. “Mammal”, as that term is used herein, refers to any animal classified as a mammal, including humans, domestic and farm animals, and zoo, sports, or pet animals, such as dogs, horses, cats, cattle, etc., Preferably, the mammal is a human.

The subject pharmaceutical compositions may be administered to a patient in any acceptable manner that is medically acceptable including orally, parenterally or with locoregional therapeutic approaches such as e.g. implants. Parenteral administration includes administering the constituents of the combined preparation by subcutaneous, intramuscular, intradermal, intramammary, intravenous injections and other administrative methods known in the art. Implants include intra arterial implants, for example, an intrahepatic artery implant.

Any of the combinations of a morpholinyl anthracycline derivative having formula (I) as defined above, and an antibody inhibiting growth factor or its receptor as listed above, are intended as fixed combination and for simultaneous, separate, or sequential use.

By the term “antineoplastic effect”, as used herein, it is meant the inhibition of the growth tumor, preferably the complete regression of the tumor, by administering an effective amount of the combination comprising a morpholinyl anthracycline derivative having formula (I), and an antibody inhibiting a growth factor or its receptor.

A further aspect of the present invention relates to the use of a combination of a morpholinyl anthracycline derivative having formula (I), as defined above, and an antibody inhibiting growth factor or its receptor as listed above, for the preparation of a medicament for the prevention or treatment of metastasis or the treatment of tumors by inhibition of angiogenesis.

The constituents of the combined preparations according to the invention can be administered to a patient in any acceptable manner that is medically acceptable including orally, parenterally, or with local therapeutic approaches such as, e.g., implants. Oral administration includes administering the constituents of the combined preparation in a suitable oral form such as, e.g., tablets, capsules, lozenges, suspensions, solutions, emulsions, powders, syrups and the like. Parenteral administration includes administering the constituents of the combined preparation by subcutaneous, intravenous or intramuscular injections. Local therapeutic approaches include implants, for example intra-arterial implants.

Typically, a morpholinyl anthracycline derivative having formula (I) is administered intravenously, typically an antibody inhibiting a growth factor or its receptor is administered intravenously or orally. The actual preferred dosage, method, order and time of administration of the constituents of the combined preparations of the invention may vary according to, inter alia, the particular pharmaceutical formulation of a morpholinyl anthracycline derivative having formula (I), being utilized and the particular pharmaceutical formulation of an antibody inhibiting a growth factor or its receptor being utilized, the particular cancer being treated, the age, condition, sex and extent of the disease treated and can be determined by one of skill in the art.

The dosage regimen must therefore be tailored to the particular of the patient's conditions, response and associate treatments, in a manner, which is conventional for any therapy, and may need to be adjusted in response to changes in conditions and/or in light of other clinical conditions.

As a non limiting example, suitable dosages of the morpholinyl anthracycline derivative of formula (I) may range from about 0.05 mg/m² to about 100 mg/m² of body surface area and, more preferably, from about 0.1 to about 10 mg/m² of body surface area. For the administration of an antibody inhibiting a growth factor or its receptor, according to the method of the invention, the course of therapy generally employed may be from 0.1 mg/kg to 100 mg/kg. More preferably, the course of therapy employed is from about 1 mg/kg to 20 mg/kg.

When the active constituents of the combined preparation according to the invention are supplied along with a pharmaceutically acceptable carrier or excipient, a pharmaceutical composition is formed. Such pharmaceutical composition constitutes a further embodiment of the invention.

Pharmaceutically acceptable carriers and excipients are chosen such that side effects from the pharmaceutical compound are minimized and the performance of the compound is not cancelled or inhibited to such an extent that treatment is ineffective. Pharmaceutically acceptable carriers or excipients to be utilized in the preparation of a pharmaceutical composition according to the invention are well known to people skilled in the art of formulating compounds in a form of pharmaceutical compositions. For example, “pharmaceutically acceptable carrier” refers to one or more compatible solid or liquid filler, diluent or encapsulating substances which are suitable for administration to mammals including humans. For example, “pharmaceutically acceptable excipient” refers to any inert substance used as a diluent or vehicle for an active substance(s) that is intentionally added to the formulation of a dosage form. The term includes binders, fillers' disintegrants, and lubricants.

Techniques for formulation and administration of drugs can be found in “Remington's Pharmacological Sciences”; Mack Publishing Co., Easton, Pa., latest edition. Pharmaceutical compositions suitable for parenteral administration are formulated in a sterile form. The sterile composition thus may be a sterile solution or suspension in a non-toxic parenterally acceptable diluent or solvent.

The amount of an active ingredient contained in the pharmaceutical composition according to the invention may vary quite widely depending upon many factors such as, for example, the administration route and the vehicle.

As an example, the pharmaceutical compositions of the invention may contain from about 0.05 mg/m² to about 100 mg/m² of body surface area of a morpholinyl anthracycline derivative of formula (I); and from 0.1 mg/kg to 100 mg/kg of an antibody inhibiting a growth factor or its receptor.

Pharmaceutical compositions according to the invention are useful in anticancer therapy. The present invention further provides a commercial kit comprising, in a suitable container means, a morpholinyl anthracycline of formula (I), as defined above, and an antibody inhibiting growth factor or its receptor. In a kit according to the invention a morpholinyl anthracycline derivative of formula (I), as defined above, and an antibody inhibiting growth factor or its receptor are present within a single container means or within distinct container means.

Another embodiment of the present invention is a commercial kit comprising a pharmaceutical composition as described above.

Kits according to the invention are intended for simultaneous, separate or sequential use in antitumor therapy.

Kits according to the invention are intended for use in anticancer therapy.

The antineoplastic effect of the combined preparations of the present invention is shown, for instance, by the following in vivo test, which is intended to illustrate the present invention without posing any limitation to it.

### EXAMPLE 1

### In Vivo Antitumor Efficacy of Nemorubicin in Combination With Bevacizumab

Materials and Methods: Balb Nu/Nu, male mice (athymic mice from Harlan, Italy) were maintained in cages with paper filter covers, food and bedding sterilized and water acidified. Human prostate carcinoma DU145 cells (from American Type Culture Collection) were implanted subcutaneously in athymic mice (2.5×10⁶/cells mice). This tumor model was selected because it was previously demonstrated that bevacizumab is able to inhibit angiogenesis and tumor growth of this experimental model [see for reference, The Prostate 36:1-10, 1998]. Drug treatments were performed on advanced tumors (when the tumors were palpable). Nemorubicin and bevacizumab were prepared immediately before use ad administered to mice in a volume of 10 ml/kg. Nemorubicin was administered as hydrochloride salt intravenously at a dose of 0.065 mg/kg and treatments were repeated weekly for 3 weeks. Bevacizumab was administered intraperitoneally at a dose of 20 mg/kg on the days 9, 13, 17, 21, 25 and 29 from the day of cell injection. When both compounds were administered on the same day, bevacizumab was administered intraperitoneally immediately before the intravenous injection of nemorubicin. Tumor growth and net body weight were evaluated every 3 days. Tumor growth was assessed by caliper. The two diameters were recorded, and the tumor weight was calculated according to the following formula: length (mm)×width² (mm)/2. The effect of the antitumor treatment was determined as the delay in the onset of an exponential growth of tumors [see for reference, Anti Cancer Drugs 7: 437-60, 1996]. This delay (T-C value) was defined as the difference of the time (in days) required for the treatment group (T) and the control group (C) tumors to reach a predetermined size (e.g. 1 g). Toxicity was evaluated on the basis of the body weight reduction.

Results The results were shown in Table 2. Nemorubicin combined with bevacizumab produced a strong synergistic effect: the T-C was significantly higher than that expected by the simple addition of the T-Cs obtained with the two compounds as single agent (17.39 days when the expected T-C was 11.72 days). No severe toxicity was observed within any treatment group.

TABLE 2
In vivo antitumor efficacy of nemorubicin
in combination with bevacizumab

| | Time to reach | T-C | Expected T-C |
|---|---|---|---|
| Treatment | 1 g (days) | (days) | (days) |
| Control (drug vehicle) | 14.27 | — | — |
| Bevacizumab 20 mg/kg* | 21.31 | 7.04 | — |
| Nemorubicin 0.065 mg/kg** | 18.95 | 4.68 | — |
| Bevacizumab 20 mg/kg + | 31.66 | 17.39 | 11.72 |
| Nemorubicin 0.065 mg/Kg*** | | | |

*Treatments administered intraperitoneally on day 9, 13, 17, 21, 25 and 29
**Treatments administered intravenously on day 9, 16 and 23 (q 7 d × 3)
***Day 9: bevacizumab was injected intraperitoneally and, immediately after, nemorubicin was injected intravenously.

1. A combination comprising a morpholinyl anthracycline derivative having formula (I): 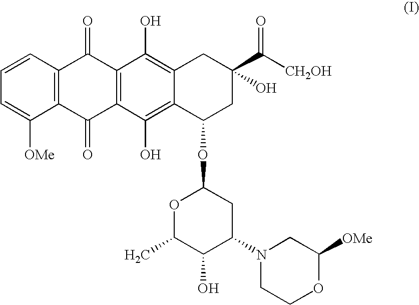
or a pharmaceutically acceptable salt thereof and an antibody inhibiting a growth factor or its receptor.

2. A combination according to claim 1, wherein the morpholinyl anthracycline of formula (I) is nemorubicin hydrochloride.

3. A combination according to claim 1 wherein the antibody inhibiting growth factor or its receptor is Bevacizumab, Cetuximab, Panitumumab, Matuzumab, Nimotuzumab, Trastuzumab or Pertuzumab.

4. A combination according to claim 1 wherein the antibody inhibiting growth factor or its receptor is Bevacizumab.

5. A pharmaceutical composition comprising a combination comprising a morpholinyl anthracycline derivative having formula (I): 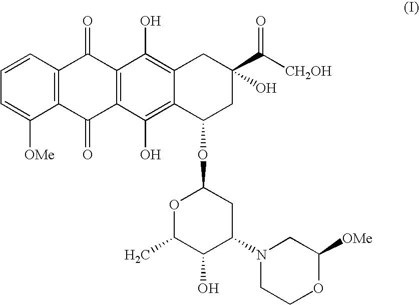
or a pharmaceutically acceptable salt thereof and an antibody inhibiting a growth factor or its receptor admixed with a pharmaceutically acceptable carrier, diluent or excipient.

6. A pharmaceutical product comprising a morpholinyl anthracycline derivative as defined in claim 1 and an antibody inhibiting a growth factor or its receptor as a combined preparation for simultaneous, sequential or separate use in therapy.

7. A combination according to claim 1 for treating a proliferative disorder.

8. A combination according 7, wherein the treatment comprises simultaneously, sequentially or separately administering the morpholinyl anthracycline derivative and an antibody inhibiting a growth factor or its receptor to a subject.

9. A commercial kit comprising, in a suitable container mean, a morpholinyl anthracycline derivative as defined in claim 1, and an antibody inhibiting growth factor or its receptor.

10. A commercial kit comprising a pharmaceutical composition or product as defined in claim 9.

11. A kit according to claim 9 for simultaneous, separate or sequential use in antitumor therapy.
